(19) United States
(12) Patent Application Publication
(10) Pub. No.: US 2006/0217405 A1
(43) Pub. Date: Sep. 28, 2006

(54) Interstitial cystitis treatment

(75) Inventors: Bobby W. Sandage JR., Lexington, MA (US); Glenn L. Cooper, Lexington, MA (US)

Correspondence Address: FOLEY AND LARDNER LLP;SUITE 500, 3000 K STREET NW, WASHINGTON, DC, 20007, US

(73) Assignee: Indevus Pharmaceuticals, Inc.

(21) Appl. No.: 11/376,555

(22) Filed: Mar. 16, 2006

Related U.S. Application Data
(60) Provisional application No. 60/662,553, filed on Mar. 17, 2005.

(51) Int. Cl. A61K 31/4747

(52) U.S. Cl. 514/278

(57) ABSTRACT

An anti-muscarinic agent exemplified by trospium is administered by intravesicular instillation into the bladder and used to treat interstitial cystitis. The instilled medication does not interact systemically with the patient; the localized effect of the medication avoids any question of systemic side effects or untoward reactions with body organs or systems.

### This application claims priority of U.S. Provisional Application No. 60/662,553, filed Mar. 17, 2005, the entire disclosure of which is incorporated herein by reference in its entirety.

### BACKGROUND OF THE INVENTION

Interstitial cystitis, one of the chronic pelvic pain disorders, is a condition resulting in recurring discomfort or pain in the bladder and the surrounding pelvic region. The symptoms of interstitial cystitis vary from case to case and even in the same individual. People may experience mild discomfort, pressure, tenderness, or intense pain in the bladder and pelvic area. Symptoms may or may not include an urgent need to urinate, frequent need to urinate, or a combination of these symptoms. Pain may change in intensity as the bladder fills with urine or as it empties. Women's symptoms often get worse during menstruation.

The bladder wall of the patient suffering from interstitial cystitis may be irritated and become scarred or stiff. Glomerulations (pinpoint bleeding caused by recurrent irritation) may appear on the bladder wall. The bladders of some interstitial cystitis sufferers have decreased urine capacity, which increases the frequency of the need to urinate. Frequency, however, is not always specifically related to bladder size; many patients with severe frequency have normal bladder capacity. Patients with severe cases of interstitial cystitis may urinate as many as 60 times a day.

Interstitial cystitis is far more common in women than in men. Of the more than 700,000 Americans estimated to have interstitial cystitis, 90 percent are women. Symptoms usually begin between 20 and 50 years of age, but the average age of onset is 40. Although only 25% of cases involve people under age 30, the number of children affected by interstitial cystitis may be greater than commonly believed.

Some of the symptoms of interstitial cystitis resemble those of bacterial infection, but medical tests reveal no organisms in the urine of patients with interstitial cystitis. Furthermore, patients with interstitial cystitis do not respond to antibiotic therapy. Researchers are working to understand the causes of interstitial cystitis and to find effective treatments.

Interstitial cystitis is a poorly understood disease with unknown causes. Although no bacteria or viruses (pathogens) have been found in the urine of interstitial cystitis sufferers, an unidentified infectious agent may be the cause. Others believe that interstitial cystitis occurs with ischemia (tissue death) or a deficiency of a protein called glycoaminoglycan (GAG) in the epithelium. The natural lining of the bladder (epithelium) is protected from toxins in the urine by a layer of the GAG protein. It has been suggested that this inner lining of the bladder may be “leaky,” allowing substances in the urine to penetrate the bladder wall. The bladder then becomes inflamed and tender and does not store urine well. This may trigger interstitial cystitis symptoms. It may be an autoimmune disease, in which the immune system attacks healthy cells, perhaps following a bladder infection. Spasms of the pelvic floor muscles may also contribute to the interstitial cystitis symptoms. It is likely that several factors cause the condition.

One theory being studied is that interstitial cystitis is an autoimmune response following a bladder infection. Another theory is that a bacterium may be present in bladder cells but not detectable through routine urine tests. Some scientists have suggested that certain substances in urine may be irritating to people with interstitial cystitis, but no substance unique to people with interstitial cystitis has as yet been isolated.

Researchers are beginning to explore the possibility that heredity may play a part in some forms of interstitial cystitis. In a few cases, interstitial cystitis has affected a mother and a daughter or two sisters, but it does not commonly run in families. No gene has yet been implicated as a cause.

Unlike inflammation of the bladder caused by bacterial infection (cystitis), which is associated with urinary tract infections (UTI) and usually treated with antibiotics, no infectious agent has been found in interstitial cystitis. Though not curable, interstitial cystitis is treatable and most patients find some relief with treatment and lifestyle changes.

In some patients, mast cells, which are associated with inflammation, are found within the bladder's mucous lining. Yet another theory is that the disorder may be an allergic reaction.

Because interstitial cystitis varies so much in symptoms and severity, most researchers believe that it is not one, but several, diseases. In the past, cases were mainly categorized as ulcerative interstitial cystitis or non-ulcerative interstitial cystitis, based on whether ulcers had formed on the bladder wall. But the vast majority of cases do not involve ulcers, and their presence or absence does not influence treatment options as much as other factors do.

For some interstitial cystitis patients, frequency of urination is their most troubling symptom. Other interstitial cystitis patients experience bladder spasms and resulting pain as a main component of their interstitial cystitis symptoms. For others, occasional urinary tract infections intensify the symptoms of interstitial cystitis.

Factors that influence treatment options include whether bladder capacity under anesthesia is great or small, and whether mast cells are present in the tissue of the bladder wall, which may be a sign of an allergic or autoimmune reaction. In some cases, the success or failure of a treatment helps characterize the type of interstitial cystitis. For example, some cases respond to changes in diet while others do not.

There is no definitive test to identify interstitial cystitis. Because symptoms are similar to those of other disorders of the urinary system such as urinary tract or vaginal infections, bladder cancer, bladder inflammation or infection caused by radiation to the pelvic area, eosinophilic and tuberculous cystitis, kidney stones, endometriosis, neurological disorders, sexually transmitted diseases, low-count bacteria in the urine, and, in men, chronic bacterial and nonbacterial prostatitis, diagnosis is sometimes difficult.

The diagnosis of interstitial cystitis in the general population is based on presence of urgency, frequency, or pelvic/bladder pain cystoscopic evidence (under anesthesia) of bladder wall inflammation, including Hunner's ulcers or glomerulations (present in 90 percent of patients with interstitial cystitis) and the absence of other diseases that could cause the symptoms

Diagnostic tests that help identify other conditions include urinalysis, urine culture, cystoscopy, biopsy of the bladder wall, distention of the bladder under anesthesia, urine cytology, and, in men, laboratory examination of prostate secretions.

It has proven impossible to predict who will respond best to which treatment. Symptoms may disappear without explanation or coincide with an event such as a change in diet or treatment. Even when symptoms disappear, they may return after days, weeks, months, or years.

Because the causes of interstitial cystitis are unknown, current treatments are aimed at relieving symptoms. Most people are helped for variable periods by one or a combination of treatments.

Many patients have noted an improvement in symptoms after a bladder distention has been done to diagnose interstitial cystitis. The procedure is now often used as one of the first treatment attempts. Symptoms may temporarily worsen 24 to 48 hours after distention, but typically return to predistention levels or improve after 2 to 4 weeks.

A number of untraditional therapies, such as acid-restricted diets, alkalization of urine, bladder holding and retraining (delaying voiding for increasingly longer intervals), biofeedback and electric stimulation, acupuncture, muscle relaxants, antidepressants, anti-inflammatories, antihistamines and analgesics, and an experimental bladder “wash” consisting of an anesthetic, an antibiotic, an anticoagulant, and hydrocortisone have been attempted with negative or mixed results.

Current medical intervention utilizes orally administration and bladder instillation as delivery methods for the few medicaments currently utilized for interstitial cystitis treatment.

Aspirin and ibuprofen are a first line of defense against mild discomfort caused by interstitial cystitis. Doctors may recommend other analgesics to relieve pain.

This first oral drug developed for treatment of the symptoms of interstitial cystitis was pentosan polysulfate sodium (Elmiron) approved by the FDA in 1996. In clinical trials, the drug improved symptoms in 38 percent of patients treated. Doctors do not know exactly how it works, but one theory is that it may repair defects that might have developed in the lining of the bladder. Elmiron's side effects are primarily gastrointestinal discomfort, although some patients experienced hair loss. However, Elmiron may also affect liver function, which therefore must be monitored.

Some patients have experienced improvement in their urinary symptoms by taking antidepressants or antihistamines. Antidepressants help reduce pain and may also help patients deal with the psychological stress that accompanies living with chronic pain. In patients with severe pain, narcotic analgesics such as acetaminophen (Tylenol) with codeine or longer acting narcotics may be necessary.

From 40 to 60 percent of interstitial cystitis patients may benefit from low doses of the tricyclic antidepressant amitriptyline (Elavil and others), according to Vicki Ratner, M.D., and colleagues in the Journal of Women's Health, Vol. 1, No. 1, 1992.

When pain is severe, some people may benefit from transcutaneous electrical nerve stimulation (TENS). Mild electrical impulses delivered to the body through wires placed on the lower back or abdomen or through devices implanted in the body may alter nerve transmissions to the bladder and help trigger release of pain-blocking hormones.

Bladder instillation, also called a bladder wash or bath, administers a fluid to the bladder. The solution is held in the bladder for varying periods of time, averaging 10 to 15 minutes, before being emptied.

In 1978, the Food and Drug Administration approved Rimso-50, a purified form of the industrial solvent dimethyl sulfoxide (DMSO, RIMSO-50), for symptomatic relief of interstitial cystitis. DMSO is the only drug approved by the U.S. Food and Drug Administration (FDA) for bladder instillation for the treatment of interstitial cystitis. DMSO treatment involves guiding a catheter up the urethra into the bladder. A measured amount of DMSO is passed through the catheter into the bladder, where it is retained for about 15 minutes before being expelled. Treatments are given weekly or biweekly for a period of 6 to 8 weeks and repeated as needed. Most people who respond to DMSO notice improvement 3 or 4 weeks after the first 6- to 8-week cycle of treatments.

For some patients, Rimso-50 treatments become less effective over time. About 50 percent of patients experience significant pain relief for an average of about 10 months. The drug works by penetrating the bladder wall to reduce inflammation and acts as a muscle relaxant by preventing muscle contractions that cause pain, frequency and urgency.

DMSO is thought to work in several ways. Because it passes into the bladder wall, it may reach tissue more effectively to reduce inflammation and block pain. It may also prevent muscle contractions that cause pain, frequency, and urgency.

Highly motivated patients who are willing to catheterize themselves may, after consultation with their doctor, be able to have DMSO treatments at home. Self-administration is less expensive and more convenient than treatment in the doctor's office.

A bothersome but relatively insignificant side effect of DMSO treatments is a garlic-like taste and odor on the breath and skin that may last up to 72 hours after treatment. Long-term treatment has caused cataracts in animal studies, but this side effect has not appeared in humans. Some patients may develop a chemical cystitis after use of the drug that goes away within one or two days. Patients taking Rimso-50 also require a blood test every six months to make sure the blood count and liver and kidney function are normal. Periodic ophthalmologic examinations are also recommended. Blood tests, including a complete blood count and kidney and liver function tests, should be done about every 6 months.

Given the constellation of symptoms of interstitial cystitis patients, an agent like trospium may provide some relief. However, oral anticholinergic agents are burdened by the typically anticholinergic side effects of dry mouth and constipation which can be troublesome for interstitial cystitis patient with co-existing conditions such as Sjogren's Syndrome. In addition, oral antichloinergic agents may not work for all interstitial cystitis patients because they can cause urinary retention (difficulty in urinating), which may already be a problem for some interstitial cystitis patients.

Trospium chloride, available in Europe for more than 20 years and under review by the US Food and Drug Administration for oral administration for the treatment of overactive bladder, is a quaternary amine that is minimally metabolized, not highly protein-bound, and theoretically should not cross the blood brain barrier. Some of the characteristics of this unique anticholinergic agent are reviewed in an article Trospium Chloride: A Quaternary Amine with Unique Pharmacologic Properties by Raymond W Pak MD, Steven P Petrou MD and David R Staskin MD, Department of Urology, 4500 San Pablo Road, Mayo Clinic, Jacksonville, Fla., 32224, USA, Current Urology Reports 2003, 4:436-440, Dec. 1, 2003.

Trospium works by blocking cholinergic receptors that are found on muscle cells in the wall of the bladder. Normally, acetylcholine acts on these receptors, under the body's control, and this causes the bladder muscle to contract and the bladder to empty. Sometimes the bladder muscle can contract uncontrollably, causing the bladder to empty too frequently or unexpectedly.

Trospium blocks the cholinergic receptors on the bladder wall, and prevents the action of acetylcholine, relaxing the bladder muscle and helps make the bladder more stable, thus relieving symptoms of bladder instability, such as frequent need to go to the toilet, sudden unexpected urges to go to the toilet, or not making it in time (incontinence).

As trospium blocks the cholinergic receptors on the bladder wall, it prevents the action of acetylcholine. This relaxes the bladder muscle and helps make the bladder more stable, thus relieving symptoms of bladder instability, such as frequent need to go to the toilet, sudden unexpected urges to go to the toilet, or not making it in time (incontinence).

Sanctura™ (trospium chloride) is a member of the class of drugs known as antimuscarinic/anticholinergic/antispasmodics. It is co-marketed by Esprit and Indevus for the treatment of overactive bladder where there are symptoms of urgency, urinary frequency, and urge urinary incontinence. The product is administered orally in 20 mg tablets.

Trospium chloride works in cholinergically innervated organs by acting as an acetylcholine antagonist on muscarinic receptors and by relaxing smooth muscle located in the bladder. As a result bladder contractions are reduced.

The plasma half-life of the product is about 20 hrs. It is primarily excreted through the feces (85.2%) with a low amount (5.8%) excreted through urine (tubular secretion).

Although no clinically significant interactions are reported other drugs such as morphine, digoxin, vancomycin, metformin, procainamide, tenofovir, and pancuronium, which are eliminated via tubular secretion may compete with trospium chloride for elimination.

Some recognized side effects are dry mouth, constipation, headache urinary retention and dry eyes.

It is an object of this invention to provide a new method of treatment of the symptions of interstitial cystitis.

It is an object of this invention to avoid the side effects of active ingredients having systemic activity by administering such active ingredients directly to the affected tissue.

### SUMMARY OF THE INVENTION

This invention relates to the use of an anti-muscarinic agent exemplified by trospium as a treatment for interstitial cystitis which demonstrates increased effectiveness and reduced side effects when administered by intravesicular instillation. The instilled medication does not interact systemically with the patient; the localized effect of the medication avoids any question of systemic side effects or untoward reactions with body organs or systems.

The method of administration provides the active ingredient directly to the affected tissues and functions directly on the indicated body tissue. In so doing the amount of active ingredient can be more specifically controlled versus other common modes of administration since the active is not metabolized or otherwise diverted as it traverses the patients body prior to arriving at the affected tissue. Furthermore, the timing of the application as well as the duration of the application can be directly controlled.

### DETAILED DESCRIPTION OF THE INVENTION

Trospium's activity as an antispasmodic is well documented. It has been employed by oral administration for the treatment of overactive bladder where it is used to mediate the functions of the bladder detressor muscle, the recognized cause of overactive bladder. There has been no recognition of its possible use in treating interstitial cystitis.

Intravascular instillation is a recognized mode of administration and has been used to administer DMSO to patients suffering form interstitial cystitis. DMSO is not an antimuscarinic agent and does not have a mode of operation similar to that of trospium or other antimuscarinics.

The novel treatment method of administering antimuscarinic agents by intravascular instillation to the bladder permits the use of agents which would otherwise not be acceptable because of their systemic effects on the patient or because of side reactions. It also permits the fine tuning of the quantity, concentration and duration of the application of the active ingredient to the affected tissues.

It one embodiment of the invention, the antimuscarinic agent containing medicament is provided to the patient in unit dose form prepackaged in a kit to allow the patient to self dose outside the hospital setting. This kit permits administration without the daily hospital trips heretofore required over the extended period, sometimes al long as months where the patient must travel to the hospital.

We have now surprisingly discovered that trospium chloride can be efficacious in the treatment of the symptom of interstitial cystitis, in situations where there is no detrusor muscle spasming as occurs in overactive bladder (OAB). Intravesicular pharmacotherapy provides high local drug concentrations in the bladder, avoids systemic side effects and eliminates the problem of low levels of urinary excretion with orally administered agents.

Trospium chloride is a quaternary ammonium compound that concentrates in the urine primarily unchanged and is believed to have local antispasmodic activity. In addition, intravesicular instillation of trospium chloride has demonstrated efficacy in patients with little or no absorption into the systemic circulation. Therefore the use of trospium, especially by intravesicular instillation, is a novel treatment for interstitial cystitis with the very low potential for systemic side effects.

Experiments have shown that while low dose of intravesical trospium had no direct effect on interstitial cystitis, and while carbachol decreased interstitial cystitis, a low dose of trospium completely blocked the inhibitory effect of carbachol.

The action of trospium is unique because it has selective activity on the bladder urothelium and bladder afferent nerves. The local trospium bladder effect is on the afferent nerve rather than bladder smooth muscle. The effect of intravesical administered trospium was not blocked by pre treatment of capsaicin or resiniferatoxin. This indicates the afferent nerve effect is that of A-delta and not C-fibers.

The concentration of trospium chloride should be maintained at a level to minimize discomfort by the patient. In general, it has been found that a concentration effective to alleviate the symptoms of interstitial cystitis is in the range of from about 0.01 to 1 mcg/ml, preferably from about 0.1 to 0.5 mcg/ml,

The trospium chloride is combined with other ingredients to form a stable solution designed for instillation by intravesical instillation.

The typical dose of trospium chloride delivered by intravesicular instillation and effective to alleviate the symptoms of interstitial cystitis is in the range of from about 0.05 to 3 mg per kilogram of patient body weight, preferably from about 0.1 to 1 mg/kg, and most preferably from about 0.25 to 0.5 mg/kg.

Further exemplary daily doses of trospium chloride, such as those suitable for a 50-70 kg person, include those ranging from about 0.5 to about 3.0 mg. More specifically, the doses can range from about 0.8 to about 2.5 mg, or from about 1.2 to about 2.2 mg.

When administered by intravesical instillation the dose is delivered over a period of from about from about 15 minutes to 2 hours and most preferably from about 30 minutes to 1 hour before being expelled.

The frequency of administration is typically weekly for a period of from about 1 week to 1 year since this is a chronic condition and this treatment is unlikely to be a cure. Exemplary treatment periods include but are not limited to 1 week to about 6 weeks.

Trospium for intravesicular instillation may be packaged in unit dose forms where an amount of medicament is pre-mixed into a suitable formulation in a single use container. For highly motivated patients the trospium chloride is provided in a unit dosage form, supplied in a kit with a single or multi-use catheter for dosing by the patient. In this form the patient avoids the time and effort of traveling to an administration center such as a hospital and is assured on providing the proper dosage.

For such purposes the amount of trospium delivered would be in the range of from about 3.5 to about 210 mg, preferably from about 7 to about 70 mg and most typically from about 17.5 to about 35 mg.

The following examples are intended to further illustrate the invention as as described above by providing certain embodiments. The examples are not intended to limit the invention in any way.

### EXAMPLE 1

Baseline cystometrogram (CMG) is measured in a group of 6 female SD rats (250 grams). The rats are anesthetized utilizing urethane anesthesia at a rate of 1.2 g/kg. Trospium chloride at a concentration of 0.5 mcg/ml is administered by intravesical instillation. Approximately, 5 ml are instilled. The trospium solution is maintained in the rat bladder for a period of 10-30 minutes. At the end of that period the solution is expelled and the CMG measured.

The results demonstrate that that trospium significantly suppressed acetic-acid induced bladder overactivity without affecting bladder contractions during voiding.

### EXAMPLE 2

A human female subject, age 40 and weighing approximately 70 kg, presents with a sense of urgency and frequency to urinate, although she is not incontinent. The subject experiences pain and other pelvic discomfort, however, while attempting to urinate. Trospium chloride at a concentration of 0.5 mcg/ml is administered twice daily by intravesical instillation to the subject for a total dose of 2.0 mg trospium chloride per day. The dosing regimen is continued for six (6) weeks. At that time, the subject no longer experiences pain when attempting to urinate.

### EXAMPLE 3

A human female subject, age 35 and weighing approximately 60 kg, presents with incontinence. Trospium chloride at a concentration of 0.5 mcg/ml is administered twice daily by intravesical instillation to the subject for a total dose of 1.6 mg trospium chloride per day. The dosing regimen is continued for eight (8) months. At that time, the subject experiences a significant reduction of urge, frequency and incontinence episodes.

What is claimed is:

1. A method for the treatment or prophylaxis of interstitial cystitis in a subject in need of such treatment or prophylaxis, comprising administering by intravesicular instillation to the subject an amount of an anti-muscarinic agent or pharmaceutically acceptable salt or prodrug thereof, wherein the amount of the anti-muscarinic agent or pharmaceutically acceptable salt or prodrug thereof comprises an interstitial cystitis treatment- or prophylaxis-effective amount of the anti-muscarinic agent.

2. The method of claim 1 where the anti-muscarinic agent is trospium chloride or a salt thereof.

3. The method of claim 2 where the trospium chloride is administered at a concentration that minimizes the chances of said patient experiencing a burning or other irntating sensation.

4. The method of claim 2 where the trospium chloride is administered in an amount of from about 3.5 to about 210 mg.

5. The method of claim 1 in which the amount of the anti-muscarinic agent in the bladder is from about 17.5 to about 70 mg.

6. The method of claim 2 in which the dwell time of trospium chloride in the bladder is from about 7 to about 35.

7. The method of claim 1 in which the anti-muscarinic agent is administered at least once weekly.

8. The method of claim 1 in which the anti-muscarinic agent is administered at least 30 once daily.

9. The method of claim 1 in which the anti-muscarinic agent is administered at a dose between about 0.05 mg/kg and about 3 mg/kg.

10. The method of claim 3 in which the trospium chloride is administered at a dose between about 0.1 mg/kg and about 1 mg/kg.

11. The method of claim 3 in which the trospium chloride is administered at a dose between about 0.25 mg/kg and about 0.5 mg/kg.

12. The method of claim 2 in which the trospium chloride is formulated with a pharmaceutically acceptable carrier.

13. The method of claim 12 in which the pharmaceutically acceptable carrier comprises an aqueous ethanol mixture having less than about 20% (v/v) ethanol and from about 0-1% (w/v) non-ionic detergent.

14. The method of claim 13 in which the pharmaceutically acceptable carrier further comprises physiologically compatible electrolytes.

15. The method of claim 14 in which the pharmaceutically acceptable carrier comprises physiological saline in the presence of a maximum amount of about 10% (v/v) ethanol.

16. The method of claim 15 in which the pharmaceutically acceptable carrier further comprises salts that buffer the pH of the pharmaceutically acceptable carrier within about the normal pH range of human urine.

17. The method of claim 16 in which administration is carried out over a period of at least about 30 days.

18. The method of claim 1 in which administration is carried out over a period of at least about six months to about one year.

19. A kit comprised of: an amount of an anti-muscarinic agent in a dosage formulation.

20. A kit for use in the treatment of interstitial cystitis, comprising: a trospium component in a formulation for intravascular instillation; a container housing the trospium component during storage and prior to administration; and instructions for carrying out drug administration of trospium in a manner effective to treat intravascular instillation.
